(19) United States
(12) Patent Application Publication
(10) Pub. No.: US 2023/0302069 A1
(43) Pub. Date: Sep. 28, 2023

(54) LACTIC ACID BACTERIA COMPOSITION STABLE UNDER STRONG ACIDIC CONDITIONS

(71) Applicant: MBIOMETHERAPEUTICS CO., LTD., Suwon-si (KR)

(72) Inventors: Se Chan KANG, Suwon-si (KR); Min Woo HAN, Anyang-si (KR); Jeong Eun KWON, Suwon-si (KR)

(21) Appl. No.: 18/041,191

(22) PCT Filed: Jul. 22, 2021
(86) PCT No.: PCT/KR2021/009468

§ 371 Date: Feb. 9, 2023

(30) Foreign Application Priority Data
Aug. 10, 2020 (KR) 10-2020-0099985

(51) Int. Cl. A61K 35/747; A23L 33/15; A61K 31/375; A23L 33/135; A61K 31/198

(52) U.S. Cl. CPC A61K 35/747; A23L 33/15; A61K 31/375; A23L 33/135; A61K 31/198; A23Y 2220/63

(57) ABSTRACT

Provided is a lactic acid bacteria composition that is stable under strong acid conditions caused by gastric acid, bile acid, or the like, and specifically, to a lactic acid bacteria composition containing a Lactobacillus paracasei strain and an amino acid as active ingredients. The lactic acid bacteria composition according to the present invention may survive even under strong acidic conditions caused by gastric acid or bile acid and reach the intestine alive, and thus defend against and attack harmful bacteria in the intestine, multiply, produce metabolites, and promote metabolite production, thereby activating the immune system.

### TECHNICAL FIELD

The present invention relates to a lactic acid bacteria composition that is stable under strong acid conditions caused by gastric acid, bile acid, or the like, and specifically, to a lactic acid bacteria composition containing a Lactobacillus paracasei strain and an amino acid as active ingredients.

### BACKGROUND ART

The intestine is an organ responsible for nutrient absorption and waste discharge, and is an immune organ that defends against harmful substances. It has been reported that more than 70% of the body's immune cells exist in the intestine, and thus the whole body can work smoothly only when the intestine is healthy. When the intestinal mucosa is damaged due to excessive drinking, smoking, stress, etc., pathogens and toxins are likely to invade the intestine, resulting in lowered immunity, which cause allergies, acne, ulcerative colitis, etc., and lead to autoimmune diseases in severe cases. In order to prevent this problem, it is necessary to activate beneficial bacteria in the intestine, and for this purpose, the importance of lactic acid bacteria intake has been emphasized.

However, lactic acid bacteria are easily killed by gastric acid or bile acid even when ingested, and thus it is difficult to expect the effect of lactic acid bacteria unless a very large amount of lactic acid bacteria are ingested. Therefore, it is necessary to develop a lactic acid bacteria preparation that can reach the intestine alive.

In order to improve the stability of lactic acid bacteria, Korean Patent No. 10-1731992 discloses a method of culturing lactic acid bacteria using a lactic acid bacteria culture medium containing waste sea mustard. The lactic acid bacteria cultured by this method have enhanced immune activity and enhanced resistance to gastric acid, bile acid and heat.

In addition, Korean Patent No. 10-2009732 discloses a method for producing lactic acid bacteria with a protein-polysaccharide double coating, which has excellent freeze-drying survival rate, acid resistance, and bile resistance.

### DISCLOSURE

### Technical Problem

The present invention is intended to provide a lactic acid bacteria that is not easily killed by gastric acid or bile acid. In addition, the present invention is intended to provide a lactic acid bacteria composition that may be taken with a vitamin. In addition, the present invention is intended to provide a lactic acid bacteria composition that may be utilized in probiotics, and furthermore, health functional foods.

### Technical Solution

The above object is achieved by a lactic acid bacteria composition containing a Lactobacillus paracasei JS1 strain (accession number: KCCM12288P) and an amino acid as active ingredients.

Preferably, the amino acid may be at least one selected from the group consisting of lysine, arginine and glutamate.

Preferably, the amino acid may be a mixture of 3 to 5 mg/g of lysine, 0.1 to 0.3 mg/g of arginine, and 0.05 to 0.2 mg/g of glutamate.

Preferably, the lactic acid bacteria composition may further contain vitamin C.

The object of the present invention is achieved by a health functional food containing the above-described lactic acid bacteria composition.

### Advantageous Effects

The lactic acid bacteria composition according to the present invention may survive even under strong acidic conditions caused by gastric acid or bile acid and reach the intestine alive, and thus defend against and attack harmful bacteria in the intestine, multiply, produce metabolites, and promote metabolite production, thereby activating the immune system. In addition, the lactic acid bacteria composition of the present invention may be taken together with a vitamin, and thus it is possible to eliminate the inconvenience of taking the composition and the vitamin at different times or separately.

### BRIEF DESCRIPTION OF DRAWINGS

FIG. 1 shows the gadC gene expression level in the lactic acid bacteria composition according to the present invention.

### MODE FOR INVENTION

All technical terms used in the present invention have the following definitions unless otherwise defined, and have the same meanings commonly understood by those of ordinary skill in the art to which the present invention pertains. In addition, preferred methods or samples are described in the present specification, but those similar or equivalent thereto are also included in the scope of the present invention.

Throughout the present specification, it should be understood that, unless the context requires otherwise, the terms “contain” and “containing” is meant to include the stated step or component, or group of steps or components, but does not exclude any other step or component, or group of steps or components.

The present invention is directed to a lactic acid bacteria composition containing lactic acid bacteria and an amino acid as active ingredients.

The lactic acid bacteria may be at least one strain selected from the group consisting of Streptococcus sp., Lactococcus sp., Enterococcus sp., Lactobacillus sp., Pediococcus sp., Leuconostoc sp., Weissella sp., and Bifidobacterium sp. Preferably, the lactic acid bacteria is a Lactobacillus paracasei JS1 strain (accession number: KCCM12288P, deposit date: Jul. 13, 2018, depository authority: Korean Culture Center of Microorganisms (International)).

According to one embodiment of the present invention, the lactic acid bacteria composition contains an amino acid, wherein the amino acid may be at least one selected from the group consisting of lysine, arginine, and glutamate. Preferably, the amino acid may be a mixture of 3 to 5 mg of lysine, 0.1 to 0.3 mg of arginine and 0.05 to 0.2 mg of glutamate, per 1 g of the composition. More preferably, the amino acid may be a mixture of 3.65 mg of lysine, 0.21 mg of arginine and 0.13 mg of glutamate, per 1 g of the composition.

According to the present invention, a mixture of lysine, arginine and glutamate is used, which may protect the growth of lactic acid bacteria from gastric acid or bile acid through proton inhibitory action, thereby increasing the number of lactic acid bacteria reaching the intestine alive. The microorganism expresses the gadC gene using the amino acid in order to overcome extreme acidic conditions such as a gastric acid environment. The present invention is characterized in that glutamate, lysine and arginine among amino acids are contained in the composition in order to increase the expression of the gadC gene under acidic conditions. In the present invention, glutamate, lysine, and arginine contained in the composition may equalize hydrogen ions outside the microorganism to hydrogen ions inside the microorganism, thereby inhibiting the death of the microorganism by osmotic pressure.

In addition, the lactic acid bacteria composition according to the present invention may further contain vitamin C or be taken together with vitamin C. Since existing lactic acid bacteria are killed by strong acid, it has to be taken at a different time from vitamin C. However, even if the lactic acid bacteria composition of the present invention is taken together with a vitamin, the lactic acid bacteria are not killed under strong acid conditions due to the proton action of the microorganism itself, and the multiplying ability of lactic acid bacteria may increase. In addition, when the composition is taken with a vitamin, secretion of gastric acid may be inhibited, and heartburn due to taking the vitamin may be prevented.

The lactic acid bacteria composition according to the present invention may be in a liquid form, a granular form, a tablet form, or the like, and may be contained in health functional foods, fermented milk, functional beverages, or the like.

Hereinafter, the present invention will be described in detail with reference to examples. However, these examples are intended to explain the present invention in more detail, and the scope of the present invention is not limited to these examples.

### Experimental Example 1: Measurement of Number of Lactic Acid Bacteria Resulting from Addition of Amino Acids

One colony of the Lactobacillus paracasei JS1 strain (accession number: KCCM12288P) was inoculated into a GAM broth medium, and then cultured in a CO₂ incubator overnight at 37° C. A new GAM broth medium (pH 2) with an extremely acidic condition was prepared by adding hydrochloric acid, and the cultured strain was inoculated into the medium in an amount of 1% based on the weight of the medium, and cultured in a CO₂ incubator at 37° C. for 1 hour.

In this case, inoculation of the medium was performed as follows: inoculation with Lactobacillus paracasei alone (Comparative Example 1); mixing of the medium with Lactobacillus paracasei and each of lysine, arginine and glutamate (Examples 1 to 3); mixing the medium with Lactobacillus paracasei and two of the amino acids (Examples 4 to 6); and mixing the medium with Lactobacillus paracasei and the three amino acids (Example 7).

After 1 hour, 0.1 ml of the strain obtained from each of the Comparative Example and the Examples was spread on a plate medium and then further cultured overnight, followed by counting the number of cells of the Lactobacillus paracasei JS1 strain. The results are shown in Table 1 below.

TABLE 1

| Items | Cell number of strain |
|---|---|
| Comparative Example 1 (Lactobacillus | 6.0 × 10² cells/plate |
| paracasei JS1) | |
| Example 1 (L. paracasei + 3.653 mg/g lysine) | 7.0 × 10³ cells/plate |
| Example 2 (L. paracasei + 0.21 mg/g arginine) | 8.2 × 10³ cells/plate |
| Example 3 (L. paracasei + 0.132 mg/g | 7.3 × 10³ cells/plate |
| glutamate) | |
| Example 4 (L. paracasei + 3.653 mg/g lysine + | 7.6 × 10³ cells/plate |
| 0.21 mg/g arginine) | |
| Example 5 (L. paracasei + 3.653 mg/g lysine + | 7.1 × 10³ cells/plate |
| 0.132 mg/g glutamate) | |
| Example 6 (L. paracasei + 0.21 mg/g | 8.0 × 10³ cells/plate |
| arginine + 0.132 mg/g glutamate) | |
| Example 7 (L. paracasei + 3.653 mg/g lysine + | 4.8 × 10⁴ cells/plate |
| 0.21 mg/g arginine + 0.132 mg/g glutamate) | |

Referring to Table 1, when the medium was mixed with one or two of the amino acids, the cell number of the strain increased by at least 10 times compared to when the medium was inoculated with Lactobacillus paracasei alone. In addition, the cell number of strains significantly increased when the medium was mixed with the three amino acids (lysine, arginine and glutamate) compared to when the medium was mixed with one or two of the amino acids.

### Experimental Example 2: Measurement of Number of Lactic Acid Bacteria Depending on Vitamin Concentration

One colony of the Lactobacillus paracasei JS1 strain (accession number: KCCM12288P) was inoculated into a GAM broth medium, and then cultured in a CO₂ incubator overnight at 37° C. A new GAM broth medium (pH 2) was prepared, and the cultured strain was inoculated into the medium in an amount of 1% based on the weight of the medium. Then, three amino acids (3.653 mg/g lysine, 0.21 mg/g arginine and 0.132 mg/g glutamate) were added to the medium, followed by culture in a CO₂ incubator at 37° C. for 1 hour. In this case, before culture, vitamin C was added to the medium in an amount of 1 wt %, 5 wt % or 25 wt % based on the weight of the medium. After 1 hour, the cell number of the Lactobacillus paracasei JS1 strain on each experimental plate was counted. The results are shown in Table 2 below.

TABLE 2

| Items | Cell number of strain |
|---|---|
| Comparative Example 1 (Lactobacillus | 6.0 × 10² cells/plate |
| paracasei JS1) | |
| Example 7 (L. paracasei + lysine + | 4.8 × 10⁴ cells/plate |
| arginine + glutamate (1)) | |
| Example 8 ((1) + 1 wt % ascorbic acid) | 6.2 × 10⁴ cells/plate |
| Example 9 ((1) + 5 wt % ascorbic acid) | 8.1 × 10⁴ cells/plate |
| Example 10 ((1) + 25 wt % ascorbic acid) | 3.9 × 10² cells/plate |

Referring to Table 2, it was confirmed that the cell number of the strain was increased by the amino acids when a mixture of the strain and the amino acid was cultured compared to the strain was cultured alone. In addition, as a result of culturing the mixture of the strain and the amino acid after adding vitamin C in an amount of 1, 5 or 25 wt % based on the weight of the medium, it was confirmed that, when vitamin C was added in an amount of 1 wt % or 5 wt % based on the weight of the medium, the acidity of the vitamin was relatively weakened by the proton action of the microorganism itself, and an environment in which the lactic acid bacteria could grow was created, thereby increasing the number of the lactic acid bacteria. It was observed that, when vitamin C was contained in a large amount of 25 wt % based on the weight of the medium, even though the acidity was lowered by the proton action of the microorganism, some of the lactic acid bacteria were killed due to a very high content of vitamin C, resulting in a decrease in the number of the lactic acid bacteria.

### Experimental Example 3: Inhibition of Gastric Acid Secretion

In order to measure the inhibition of gastric acid secretion after gastric mucosal damage, 1 mL of ethanol was orally administered to 7-week-old male SD rats for 7 days to stimulate the stomach, and at the same time, 200 μL of each test substance was orally administered to confirm whether it protected the gastric mucosa. The test substances were (1) L. paracasei+lysine+arginine+glutamate (Example 7), (2) (1)+1 wt % vitamin C (Example 8), (3) (1)+5 wt % vitamin C (Example 9), and (4) (1)+25 wt % vitamin C (Example 10).

The SD rats were fasted 12 hours before dissection to empty the stomach, and on the day of the experiment, 1 ml of 70% ethanol+150 mM HCl was orally administered to each rate. After 1 hour, the rats were euthanized. After euthanasia, the pylorus of the stomach harvested by laparotomy was incised, gastric juice was collected, and the supernatant was isolated. A 0.5% dimethylaminobenzene alcohol solution and a 1% phenolphthalein alcohol solution were added to 1 ml of the isolated supernatant, and when the supernatant appeared red, a 0.1N NaOH solution was added thereto. The titration value until a rose color appeared was defined as the total acidity and expressed in units of mEq/L. The results are shown in Table 3 below.

Acidity=volume of NaOH×normality of NaOH×100/0.1 (meq⁻¹/100 g) [Equation 1]

TABLE 3

| | Total acidity |
|---|---|
| Items | (molar equiv · L⁻¹/100 g) |
| Vehicle | 30.7 ± 5.73 |
| Control group | 79.4 ± 7.02 |
| L. paracasei + lysine + arginine + | 60.3 ± 3.06 |
| glutamate (1) | |
| (1) + 1 wt % ascorbic acid | 52.7 ± 4.32 |
| (1) + 5 wt % ascorbic acid | 34.8 ± 5.67 |
| (1) + 25 wt % ascorbic acid | 54.1 ± 8.19 |

As a result of examining the change in acidity of gastric juice that damages the gastric mucosa, it was confirmed that the total acidity in the vehicle group was 30.7±5.73 molar equiv·L⁻¹/100 g and the total acidity in the control group was 79.4±7.02 molar equiv·L⁻¹/100 g, which increased compared to that in the normal control group. In addition, the total acidity in the group treated with the lactic acid bacteria strain (L. paracasei) and the three amino acids (lysine+arginine+glutamate) slightly decreased, and the total acidity in the group treated with (1)+5 wt % vitamin C (ascorbic acid) was 34.8±5.67 molar equiv·L⁻¹/100 g, which was similar to that in the vehicle group. From these results, it can be confirmed that the lactic acid bacteria composition according to the present invention inhibits secretion of gastric acid even when the lactic acid bacteria strain, the amino acids, and the vitamin are taken together.

### Experimental Example 4: Analysis of Expression Level of Gad C Gene in Microorganism

One colony of the Lactobacillus paracasei JS1 strain (accession number: KCCM12288P) was inoculated into a GAM broth medium, and then cultured in a CO₂ incubator overnight at 37° C. A new GAM broth medium (pH 2) was prepared, and the cultured strain was inoculated into the medium in an amount of 1% based on the weight of the medium. Then, three amino acids (3.653 mg/g lysine, 0.21 mg/g arginine and 0.132 mg/g glutamate) were added to the medium, followed by culture in a CO₂ incubator at 37° C. for 1 hour. In this case, before culture, vitamin C was added to the medium in an amount of 1 wt %, 5 wt % or 25 wt % based on the weight of the medium.

After mixing and culturing, each culture was centrifuged, the strain was isolated, and RNA was isolated therefrom using the Trizol/bead method. RT-PCR was performed after treatment with DNase (RQ1 RNase-free DNase; Promega, WI, USA, WI). M-MLV reverse transcriptase (Enzynomics, Daejeon, Korea) was used, and the reaction mixture consisted of 2 μl of 10× buffer, 2 μl of dNTP mixture (2 mM each), 0.5 μl of forward primer, 0.5 μl of reverse primer, 0.5 μl of reverse transcriptase, 2 μl of RNA (1 μg), 0.5 μl of RNase inhibitor, and 12 μl of water. The reaction was incubated at 37° C. for 30 minutes, followed by initial PCR activation at 95° C. for 15 minutes. The PCR cycle consisted of denaturation at 95° C. for 0.5 min, annealing at 58° C. for 0.5 min, and extension at 72° C. for 1 min. PCR was performed for a total of 29 cycles, and final extension was performed at 72° C. for 10 minutes.

gad C forward:
5′-TCGGCCGAATAATGAGTTCCC-3′
gad C reverse:
5′-AACGGAGCCTGTGTACGTAA-3′

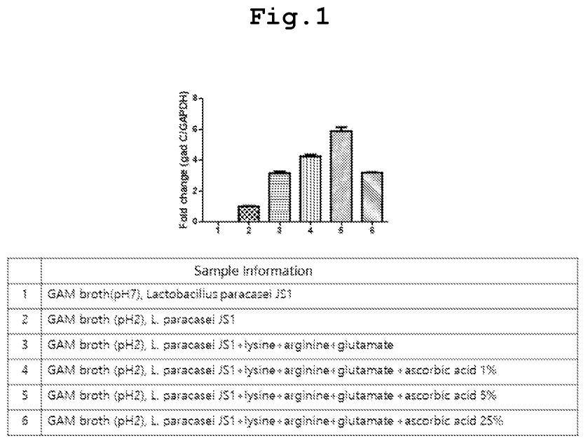

The results of the study are shown in FIG. 1.

Referring to FIG. 1, expression of the gad C gene was not observed at all when the strain was cultured alone in a general GAM broth medium, but very weak expression of the gad C gene was observed when the strain was cultured alone in a GAM broth medium (pH 2) under strong acid conditions. Expression of the gad C gene was found in the group in which the mixture of the amino acids and the lactic acid bacteria was cultured under strong acid conditions and in the experimental group in which the mixture of the lactic acid bacteria, the vitamin and the amino acids was cultured.

It was confirmed that, among these groups, the group in which the mixture of the lactic acid bacteria, the amino acid and 5 wt % vitamin C was cultured showed the highest expression of the gad C gene, and that these results were consistent with the results of Examples 1 and 2 that indicate that the number of the lactic acid bacteria increased.

1. A lactic acid bacteria composition containing a Lactobacillus paracasei JS1 strain (accession number: KCCM12288P) and an amino acid as active ingredients.

2. The lactic acid bacteria composition according to claim 1, wherein the amino acid is at least one selected from the group consisting of lysine, arginine, and glutamate.

3. The lactic acid bacteria composition according to claim 1, wherein the amino acid is a mixture of 3 to 5 mg/g of lysine, 0.1 to 0.3 mg/g of arginine, and 0.05 to 0.2 mg/g of glutamate.

4. The lactic acid bacteria composition according to claim 1, wherein the lactic acid bacteria composition further contains vitamin C.

5. A health functional food containing the lactic acid bacteria composition according to claim 1.

6. A health functional food containing the lactic acid bacteria composition according to claim 2.

7. A health functional food containing the lactic acid bacteria composition according to claim 3.

8. A health functional food containing the lactic acid bacteria composition according to claim 4.
